(19) United States
(12) Patent Application Publication
(10) Pub. No.: US 2024/0165140 A1
(43) Pub. Date: May 23, 2024

(54) MEDICATION FOR INHIBITING DNA-PKCS

(71) Applicants: TSINGHUA UNIVERSITY, Beijing (CN); BEIJING PROTGEN LTD., Beijing (CN)

(72) Inventors: Yongzhang LUO, Beijing (CN); Lin JIA, Beijing (CN); Guodong CHANG, Beijing (CN)

(73) Assignees: TSINGHUA UNIVERSITY, Beijing (CN); BEIJING PROTGEN LTD., Beijing (CN)

(21) Appl. No.: 18/512,519

(22) Filed: Nov. 17, 2023

Related U.S. Application Data
(63) Continuation of application No. 16/635,006, filed on Jan. 29, 2020.

(30) Foreign Application Priority Data
Jul. 30, 2017 (CN) 201710638033.2
Aug. 24, 2017 (CN) 201710733900.0

(51) Int. Cl. A61K 31/7048; A61K 38/17; A61P 35/00

(52) U.S. Cl. CPC A61K 31/7048; A61K 38/17; A61P 35/00

(57) ABSTRACT

A method for treating a tumor or cancer by means of a treatment scheme of using endostatin in combination with induction of DNA double-strand breaks, and a medication. The method and composition are used for treating a tumor or cancer related to loss of p53 function, or a tumor or cancer occurring when p53 function is normal.

### CROSS-REFERENCE TO RELATED APPLICATIONS

This application is a continuation of U.S. application Ser. No. 16/635,006, which is the national phase of PCT application PCT/CN2018/097609 having an international filing date of Jul. 27, 2018, which claims priority of Chinese patent application Nos. 201710638033.2 filed Jul. 30, 2017, and 201710733900.0 filed Aug. 24, 2017. The contents of the above patent applications are incorporated by reference herein in their entirety.

### FIELD OF THE INVENTION

The present invention relates to methods and medicaments for treating tumor or cancer, in particular, methods and medicaments for treating tumor or cancer by inhibiting DNA-dependent protein kinase catalytic subunit (DNA-PKcs).

### BACKGROUND OF THE INVENTION

Tumor is a disease in which multiple factors play a role and are involved, and which develops in multiple stages. Chemotherapy plays an important role in the treatment of tumor. However, tumor cells are prone to develop resistance to chemotherapeutic agents, which seriously affects the treatment of cancer patients. Commonly used chemotherapeutic agents, such as alkylating agents, platinum complexes and topoisomerase inhibitors, use DNA as target and kill tumor cells by causing DNA damage to induce apoptosis. Therefore, DNA damage repair is considered to be an important reason for the resistance of tumor cells to chemotherapeutic agents. There are many forms of DNA damage caused by chemotherapeutic agents, including base damage, single-strand break (SSB), double-strand break (DSB), etc., among which DSB injury is the most serious. In mammalian cells, there are two main forms of DSB damage repair: Homologous Recombination (HR) and Nonhomologous End Joining (NHEJ). The HR repair process uses sister chromatid as template to accurately repair damaged DNA double strand, but only in the S and G2 phases of the cell cycle. Compared to HR, NHEJ is a more conservative repair mechanism that can be performed at any stage of the cell cycle. This repair does not require a homologous template, and uses protein interactions to directly connect damaged ends, which is the main mechanism for DSB repair in mammalian cells. The basic process of NHEJ is that after double-strand break occurs, Ku70/Ku80 binds to the end of the DSB injury site, and then Ku recruits DNA-PKcs to form a DNA-PK complex, which initiates the NHEJ repair mechanism. DNA-PKcs transmits repair signals through autophosphorylation and phosphorylation of downstream proteins. DNA terminal processing proteins (nucleases, polymerases, etc.) process the broken ends into structures suitable for ligation, and finally the ligase IV-XRCC4-XLF complex performs the ligation function. Among them, DNA-PKcs is the catalytic subunit of the DNA-PK complex, is a member of the phosphatidylinositol-3-kinase-like kinase family (PIKK) and plays a central role in NHEJ repair. Studies have shown that inhibition of DNA-PKcs can greatly enhance the sensitivity of tumor cells to DNA damaging agents (Elaine Willmore, et al. Blood. 2004; 103: 4659-4665, Yan Zhao, et al. Cancer Res 2006; 66(10): 5354-62). DNA-PK inhibitors are also under continuous development, and some DNA-PK inhibitors have entered clinical trials. It is a promising area to use a DNA repair inhibitor in combination with a chemotherapeutic agent to treat tumors.

P53 protein is considered to be one of the most famous tumor suppressors so far. It plays an important regulatory role in tumorigenesis and development, and is involved in multiple life activity processes such as cell cycle arrest, cell aging, DNA damage repair and apoptosis. Studies have shown that, under the stimulation of chemotherapeutic agents, wild-type P53 can activate caspase family proteins and cause tumor cell apoptosis by regulating a series of downstream target genes (Liz J Valente, et al. BioDiscovery 2013; 8: 3); however, the incidence of p53 gene abnormalities is high in tumors, including gene deletion or mutation. Abnormal p53 gene often causes loss of normal function, which in turn reduces the sensitivity of tumor cells to chemotherapeutic agents. Studies have shown that tumors with normal P53 function have a better response to chemotherapeutics agents than tumors with loss of P53 function (Toshiyuki Harada, et al. Cancer Sci 2003; 94: 394-399).

Endostatin (ES) is an endogenous neovascularization inhibitor discovered by a research group led by Professor Folkman from Harvard University in 1997. It was identified as the C-terminal fragment of collagen XVIII. It has shown activities in vitro and in vivo tests, such as inhibiting the proliferation of activated vascular endothelial cells, inhibiting the formation of new blood vessels, and inhibiting the development and metastasis of tumors, without cytotoxicity and developing drug resistance (O'Reilly M S et al. Cell 1997; 88:277-285; Boehm T. et al. Nature 1997; 390: 404-407). Subsequent studies have found that Endostatin also has a variety of biological activities, including inhibition of lymphatic endothelial cell proliferation, migration and lymphangiogenesis, inhibition of adipocyte differentiation and insulin resistance, etc. (Zhuo W et al. J Pathol 2010; 222, 249-260, Wang H et al. Diabetes 2015; 64, 2442-2456). But so far, there have been no reports showing that Endostatin regulates DNA damage repair pathways.

### SUMMARY OF THE INVENTION

The present invention provides a method and a medicament for treating tumor or cancer by using an endostatin in combination with a treatment regimen for inducing DNA double-strand break. The method and composition of the present invention can be used to treat tumor or cancer with loss of P53 function, or tumor or cancer with normal P53 function.

According to one aspect of the present invention, a pharmaceutical composition is provided, comprising an endostatin and a chemotherapeutic agent, wherein the chemotherapeutic agent is a chemotherapeutic agent that induces DNA double-strand break.

In some embodiments, the pharmaceutical composition further comprises pharmaceutically acceptable carrier.

In some embodiments, the chemotherapeutic agent is Etoposide or Doxorubicin.

In some embodiments, the pharmaceutical composition is used for treating tumor or cancer.

In some embodiments, the tumor or cancer is deficient in P53 function.

In some embodiments, the tumor or cancer is normal in P53 function.

In some embodiments, the tumor or cancer is non-small cell lung cancer or melanoma.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, a combination of drugs is provided, including an endostatin and a chemotherapeutic agent, wherein the chemotherapeutic agent is a chemotherapeutic agent that induces DNA double-strand break.

In some embodiments, the chemotherapeutic agent is Etoposide or Doxorubicin.

In some embodiments, the combination of drugs is used for treating tumor or cancer.

In some embodiments, the tumor or cancer is deficient in P53 function.

In some embodiments, the tumor or cancer is normal in P53 function.

In some embodiments, the tumor or cancer is non-small cell lung cancer or melanoma.

In some embodiments, the endostatin and the chemotherapeutic agent are administered simultaneously or sequentially.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, a kit is provided, comprising: a) a first agent which is an endostatin or a pharmaceutical composition containing an endostatin; b) a second agent which is a chemotherapeutic agent, wherein the chemotherapeutic agent is a chemotherapeutic agent that induces DNA double-strand break.

In some embodiments, the chemotherapeutic agent is Etoposide or Doxorubicin.

In some embodiments, the kit is used for treating tumor or cancer.

In some embodiments, the tumor or cancer is deficient in P53 function.

In some embodiments, the tumor or cancer is normal in P53 function.

In some embodiments, the tumor or cancer is non-small cell lung cancer or melanoma.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, a method of increasing sensitivity of a cell to a treatment regimen for inducing DNA double-strand break is provided, comprising a step of contacting the cell with an endostatin, wherein the cell is deficient in P53 function.

In some embodiments, the cell is a tumor cell or cancer cell.

In some embodiments, the tumor cell or cancer cell is a non-small cell lung cancer cell or melanoma cell.

In some embodiments, the method is performed in vivo or in vitro.

In some embodiments, the step of contacting the cell with an endostatin is performed simultaneously or sequentially with the treatment regimen for inducing DNA double-strand break.

In some embodiments, the cell is contacted with an endostatin prior to the treatment regimen for inducing DNA double-strand break.

In some embodiments, the cell is contacted with an endostatin after the treatment regimen for inducing DNA double-strand break.

In some embodiments, the treatment regimen for inducing DNA double-strand break is a radiotherapy and/or administration of a chemotherapeutic agent.

In some embodiments, the chemotherapeutic agent is Etoposide or Doxorubicin.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, use of an endostatin in the preparation of a medicament in combination with a treatment regimen for inducing DNA double-strand break for the treatment of tumor or cancer is provided.

In some embodiments, the tumor or cancer is deficient in P53 function.

In some embodiments, the tumor or cancer is normal in P53 function.

In some embodiments, a therapeutic dose of the treatment regimen for inducing DNA double strand break is less than a therapeutic dose of the treatment regimen when used alone.

In some embodiments, the tumor or cancer is non-small cell lung cancer or melanoma.

In some embodiments, the treatment regimen for inducing DNA double-strand break is a radiotherapy and/or a chemotherapeutic agent.

In some embodiments, the chemotherapeutic agent is Etoposide or Doxorubicin.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, a method of treating tumor or cancer in a subject is provided, comprising:

a) performing a treatment regimen for inducing DNA double-strand break in a subject; and

b) administering an endostatin to the subject.

In some embodiments, the tumor or cancer is deficient in P53 function.

In some embodiments, the tumor or cancer is normal in P53 function.

In some embodiments, a therapeutic dose of the treatment regimen for inducing DNA double-strand break is less than a therapeutic dose of the treatment regimen when used alone.

In some embodiments, the tumor or cancer is non-small cell lung cancer or melanoma.

In some embodiments, the step of contacting a cell with an endostatin is performed simultaneously or sequentially with the treatment regimen for inducing DNA double-strand break.

In some embodiments, an endostatin is administered to the subject prior to the treatment regimen for inducing DNA double-strand break.

In some embodiments, an endothelin is administered to the subject after the treatment regimen for inducing DNA double-strand break.

In some embodiments, the treatment regimen for inducing DNA double-strand break is a radiotherapy and/or a chemotherapeutic agent.

In some embodiments, the chemotherapeutic agent is Etoposide or Doxorubicin.

In some embodiments, the subject is a human.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, a method for inducing apoptosis is provided, comprising:

a) inducing DNA double-strand break in a cell; and

b) contacting the cell with an endostatin.

In some embodiments, the cell is a tumor or cancer cell.

In some embodiments, the tumor or cancer cell is deficient in P53 function.

In some embodiments, the tumor or cancer cell is normal in P53 function.

In some embodiments, a dosage applied in the step of inducing DNA double-strand break in a cell is less than a dosage applied when the treatment regimen is used alone.

In some embodiments, the tumor or cancer is non-small cell lung cancer or melanoma.

In some embodiments, the step of inducing DNA double-strand break in a cell is performed simultaneously or sequentially with the step of contacting the cell with an endostatin.

In some embodiments, the cell is contacted with an endostatin prior to inducing DNA double-strand break in the cell.

In some embodiments, the cell is contacted with an endostatin after inducing DNA double-strand break in the cell.

In some embodiments, the DNA double-strand break is induced by irradiating the cell or contacting the cell with a chemotherapeutic agent.

In some embodiments, the chemotherapeutic agent is Etoposide or Doxorubicin.

In some embodiments, the method is performed in vitro or in vivo.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, use of an endostatin in the preparation of a medicament for inducing apoptosis is provided, wherein the cell is induced to generate a DNA double-strand break.

In some embodiments, the cell is a tumor or cancer cell.

In some embodiments, the tumor or cancer cell is deficient in P53 function.

In some embodiments, the tumor or cancer cell is normal in P53 function.

In some embodiments, the tumor or cancer is non-small cell lung cancer or melanoma.

In some embodiments, the cell is induced to generate a DNA double-strand break by radiation irradiation or exposure to a chemotherapeutic agent.

In some embodiments, the chemotherapeutic agent is Etoposide or Doxorubicin.

In some embodiments, the endostatin is:

a natural human endostatin;

an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

According to another aspect of the present invention, a method of inhibiting DNA-PKcs activity in a biological sample is provided, comprising contacting the biological sample with an endostatin.

In some embodiments, the endostatin is:

a natural human endostatin;

an endostatin variant obtained by adding 9 additional amino acids MGGSHHHHH to the N-terminus of the natural human endostatin, wherein the Met at the N-terminus of the endothelin variant is sometimes partially deleted when expressed by E. coli; or

a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

### BRIEF DESCRIPTION OF THE DRAWINGS

FIG. 1A: Coomassie blue-staining showed the Endostatin-binding proteins in tumor cells as detected by SDS-PAGE. CNBr represents hydrogen bromide-activated agarose gel 4B affinity medium.

FIG. 1B: The Endostatin-binding proteins in tumor cells as detected by immunoblotting assay.

FIG. 1C: The interaction of Endostatin with DNA-PKcs in tumor cells as detected by co-immunoprecipitation assay.

FIG. 2A: Immunoblotting assay showed that Endostatin inhibited the activation of DNA-PKcs signaling pathway in H1299 cells induced by the chemotherapeutic agent.

FIG. 2B: Immunoblotting assay showed that Endostatin inhibited the activation of DNA-PKcs signaling pathway in A549 cells induced by the chemotherapeutic agent.

FIG. 3A: Immunoblotting assay showed that Endostatin delayed DNA-PKcs-mediated DNA damage repair in H1299 cells.

FIG. 3B: Immunoblotting assay showed that Endostatin delayed DNA-PKcs-mediated repair of DNA damage in A549 cells.

FIG. 4A: Endostatin synergized with Etoposide promoted the apoptosis of H1299 cells, which depended on the presence of DNA-PKcs. ** represents P<0.01; ns, no significant difference.

FIG. 4B: Endostatin synergized with Etoposide promoted the apoptosis of A549 cells, which depended on the presence of DNA-PKcs. ** represents P<0.001; ns, no significant difference.

FIG. 5A: Endostatin synergized with Etoposide inhibited the activity of H1299-NC cells. * represents P<0.05; *** represents P<0.001.

FIG. 5B: Endostatin cannot enhance the inhibitory effect of Etoposide on the activity of H1299-P53 cells.

FIG. 5C: Endostatin cannot enhance the inhibitory effect of Etoposide on the activity of A549-NC cells.

FIG. 5D: Endostatin synergized with Etoposide inhibited the activity of A549-shP53 cells. * represents P<0.05; ** represents P<0.01.

FIG. 6A: Endostatin synergized with Etoposide inhibited the survival of H1299-NC cells. *** represents P<0.001.

FIG. 6B: Endostatin cannot enhance the inhibitory effect of Etoposide on the survival of H1299-P53 cells. ns, no significant difference.

FIG. 6C: Endostatin cannot enhance the inhibitory effect of Etoposide on the survival of A549-NC cells. ns, no significant difference.

FIG. 6D: Endostatin synergized with Etoposide inhibited the survival of A549-shP53 cells. *** represents P<0.001.

FIG. 7A: Endostatin synergized with Etoposide promoted apoptosis of H1299-NC cells. *** represents P<0.001.

FIG. 7B: Endostatin cannot enhance Etoposide to promote the apoptosis of H1299-P53 cells. ns, no significant difference.

FIG. 7C: Endostatin cannot enhance Etoposide to promote the apoptosis of A549-NC cells. ns, no significant difference.

FIG. 7D: Endostatin synergized with Etoposide inhibited the apoptosis of A549-shP53 cells. *** represents P<0.001.

FIG. 8A: Endostatin synergized Doxorubicin inhibited the activity of H1299-NC cells. * represents P<0.05; ** represents P<0.01.

FIG. 8B: Endostatin cannot enhance the inhibitory effect of Doxorubicin on the activity of H1299-P53 cells.

FIG. 8C: Endostatin cannot enhance the inhibitory effect of Doxorubicin on the activity of A549-NC cells.

FIG. 8D: Endostatin synergized Doxorubicin inhibited the activity of A549-shP53 cells. * represents P<0.05; *** represents P<0.001.

FIG. 9A: Endostatin synergized with Etoposide inhibited the activity of MDA-MB-435S cells. * represents P<0.05; ** represents P<0.01.

FIG. 9B: Endostatin cannot enhance the inhibitory effect of Etoposide on the activity of A375 cells.

FIG. 10A: Immunoblotting assay showed that Endostatin inhibited the repair of cell damage by DNA-PKcs, and promoted the activation of apoptotic pathway and degradation of DNA-PKcs in H1299-NC cells.

FIG. 10B: Immunoblotting assay showed that Endostatin had no effect on DNA damage, activation of apoptotic pathway, and degradation of DNA-PKcs in H1299-P53 cells induced by Etoposide.

FIG. 10C: Immunoblotting assay showed that Endostatin had no effect on DNA damage, activation of apoptotic pathway, and degradation of DNA-PKcs in A549-NC cells induced by Etoposide.

FIG. 10D: Immunoblotting assay showed that Endostatin inhibited the repair of cell damage by DNA-PKcs, and promoted the activation of apoptotic pathway and degradation of DNA-PKcs in A549-shP53 cells.

FIG. 11A: Endostatin synergized with Etoposide up-regulated mRNA expression levels of pro-apoptotic molecules BAX, NOXA, PUMA, P21 in H1299-NC cells. * represents P<0.05; ** represents P<0.01; *** represents P<0.001.

FIG. 11B: Endostatin had no effect on mRNA expression levels of pro-apoptotic molecules BAX, NOXA, PUMA, P21 in H1299-P53 cells up-regulated by Etoposide. ns, no significant difference.

FIG. 11C: Endostatin had no effect on the mRNA expression levels of pro-apoptotic molecules BAX, NOXA, PUMA, P21 in A549-NC cells up-regulated by Etoposide. ns, no significant difference.

FIG. 11D: Endostatin synergized with Etoposide up-regulated mRNA expression levels of pro-apoptotic molecules BAX, NOXA, PUMA, P21 in A549-shP53 cells. * represents P<0.05; ** represents P<0.01.

FIG. 12A: Endostatin enhanced the inhibitory effect of Etoposide on H1299-NC tumor growth. ** represents P<0.01.

FIG. 12B: Endostatin cannot enhance the inhibitory effect of Etoposide on H1299-P53 tumor growth. ns, no significant difference.

FIG. 12C: Endostatin cannot enhance the inhibitory effect of Etoposide on A549-NC tumor growth. ns, no significant difference.

FIG. 12D: Endostatin enhanced the inhibitory effect of Etoposide on A549-shP53 tumor growth. ** represents P<0.01.

FIG. 13A: The combined use of Endostatin and Etoposide had a synergistic effect on prolonging the survival time of H1299 tumor-bearing mice. * represents P<0.05.

FIG. 13B: The combined use of Endostatin and Etoposide cannot further prolong the survival time of A549 tumor-bearing mice in the group treated with Etoposide alone. ns, no significant difference.

FIG. 14A: Immunofluorescence method showed that Endostatin synergized with Etoposide inhibited the proliferation of H1299-NC tumor and the expression level of DNA-PKcs. * represents P<0.05; ** represents P<0.01.

FIG. 14B: Immunofluorescence method showed that Endostatin could not enhance the inhibitory effect of Etoposide on the proliferation of H1299-P53 tumor and the expression level of DNA-PKcs. ns, no significant difference.

FIG. 14C: Immunofluorescence method showed that Endostatin could not enhance the inhibitory effect of Etoposide on the proliferation of A549-NC tumor and the expression level of DNA-PKcs. ns, no significant difference.

FIG. 14D: Immunofluorescence method showed that Endostatin synergized with Etoposide inhibited the proliferation of A549-shP53 tumor and the expression level of DNA-PKcs. * represents P<0.05; ** represents P<0.01.

FIG. 15A: Immunoblotting assay showed that ZBP-Endostatin and PEG-Endostatin inhibited the activation of DNA-PKcs signaling pathway in H1299 cells induced by the chemotherapeutic agent.

FIG. 15B: Immunoblotting assay showed that ZBP-Endostatin and PEG-Endostatin inhibited the activation of DNA-PKcs signaling pathway in A549 cells induced by the chemotherapeutic agent.

### DETAILED DESCRIPTION OF THE INVENTION

It is found in the present invention that an endostatin can directly act on tumor cells and regulate the sensitivity of tumor cells to chemotherapeutic agents. It is found in in vitro cytological experiments that an endostatin can significantly enhance the inhibitory effect of chemotherapeutic agents on P53-deficient tumor cell activity and cell colony-forming ability, and promote tumor cell apoptosis induced by the chemotherapeutic agents. Meanwhile, in a mouse xenograft tumor model, an endostatin can significantly enhance the growth inhibiting effect of chemotherapeutic agents on P53-deficient non-small cell lung cancer xenografts and prolong the survival of tumor-bearing mice.

In the present invention, in order to explore the mechanism by whichan endostatin regulates the sensitivity of tumor cells to chemotherapy, by screening proteins that interact with an endostatin in tumor cells, it was found that the endostatin can specifically interact with DNA-PKcs. DNA-PKcs is a key protein in the non-homologous end joining pathway in mammalian cells and is involved in the repair of DNA double-strand breaks in cells. The interaction between an endostatin and DNA-PKcs can inhibit its activity and further inhibit DNA damage repair. P53-deficient tumor cells show resistance to chemotherapeutic agents, and cell survival depends on the DNA-PKcs-mediated DNA repair process. In this situation, endostatin causes the accumulation of DNA damage in cells by inhibiting the activity of DNA-PKcs, and then induces apoptosis and enhances the therapeutic effect of chemotherapeutic agents. Cells with normal P53 function are more sensitive to chemotherapeutic agents. P53 activates Caspase-3-mediated apoptosis under the treatment of a chemotherapeutic agent alone, and DNA-PKcs as a substrate of Caspase-3 will also be cleaved and reduced during this process. Therefore, the effect of using the endostatin to inhibit DNA-PKcs is very limited.

The action mechanism of an endostatin in P53-deficient tumor cells can be understood as follows. Since P53 can activate caspase family proteins, and cause tumor cell apoptosis by regulating a series of downstream target genes, P53-deficient tumor cells have a higher apoptosis threshold, and with the stimulation by DNA damage inducers such as Etoposide, the DNA-PKcs signaling pathway is activated and mediates DNA damage repair through non-homologous end-joining pathway. In this situation, endostatin can directly bind to DNA-PKcs and inhibit its activity, causing accumulation of DNA damage in cells, and the cells develop from DNA repair to apoptosis. At this time, the activation of Caspase pathway in turn leads to the degradation of DNA-PKcs, and further blocks DNA repair and promotes cell death. In contrast, during DNA damage, tumor cells with normal P53 function have lower apoptosis threshold, and P53 will trigger the activation of apoptosis-related effectors. In this situation, in the early stage of apoptosis, DNA-PKcs is degraded by Caspase-3 activated by the P53 pathway, which further weakens DNA repair. Therefore, the P53 signal pathway itself can form a feedback regulatory loop. The effect of increasing the chemotherapy sensitivity is not ideal by targeting endothelin to DNA-PKcs in tumor cells with normal P53 function.

Therefore, the present invention is essentially based on the discovery that an endostatin can directly bind to DNA-PKcs and inhibit its activity, that is, the endostatin is an inhibitor of DNA-PKcs. For P53-deficient cells, when DNA damage is induced by any treatment method (such as a chemotherapeutic agent or radiotherapy), endostatin can promote cell apoptosis through the above-mentioned mechanism, thereby increasing the sensitivity of cells to the treatment method. Even for cells with normal P53 function, the use of endostatin can reduce the dose of a chemotherapeutic agent or the dose of radiotherapy. At this time, although the DNA damage of cells is reduced due to the decrease in the dose of a chemotherapeutic agent or the dose of radiotherapy, the endostatin can inhibit the activity of DNA-PKcs, thereby promoting the accumulation of DNA damage in cells and promoting apoptosis. Therefore, the effect of an original dose can still be achieved when the dose of a chemotherapeutic agent or the dose of radiotherapy is reduced, thereby reducing the side effects of the chemotherapeutic agent or radiotherapy and improving its compliance.

“Endostatin (ES)” as described herein has its broadest meanings and includes a variety of proteins with natural endostatin activities, such as the activities of inhibiting the vascular endothelial cell proliferation, migration and angiogenesis in vivo.

The “endostatin” as described herein may be a natural endostatin, preferably a natural human endostatin, but is not limited thereto. For example, it may be a natural endostatin from other mammals, such as mouse, rat, pig, dog, rabbit, sheep, goat, cat, and the like. The endostatin may be purified from a natural source, or be a recombinant protein.

The sequence of the natural human endostatin used in the present invention may be, for example:

(SEQ ID NO: 2)
MHSHRDFQPVLHLVALNSPLSGGMRGIRGADFQCFQQARAVGLAGTFRAF
LSSRLQDLYSIVRRADRAAVPIVNLKDELLFPSWEALFSGSEGPLKPGAR
IFSFDGKVDLRHPTWPQKSVWHGSDPNGRRLTESYCETWRTEAPSATGQA
SSLLGGRLLGQSAASCHHAYIVLCIENSFMTASK;

wherein the Met at the N-terminus is sometimes partially deleted when it is expressed by E. coli.

The “endostatin” as described herein may also be a functional variant of natural endostatin, such as an engineered functional variant, which has substitution, deletion or addition of one or several amino acids compared with natural endostatin, and has basically the same biological functions, such as the activities of inhibiting the proliferation, migration and in vivo angiogenesis of vascular endothelial cells.

The term “functional variant” as used herein includes mutants of endostatin that contain substitutions, deletions or additions of one or more (for example, 1-5, 1-10, or 1-15, in particular, such as 1, 2, 3, 4, 5, 6, 7, 8, 9, 10, 11, 12, or more) amino acids in the amino acid sequence, and the mutants have biological activities similar to those of endostatin to inhibit the proliferation, migration and in vivo angiogenesis of vascular endothelial cells. The biological activities of a “functional variant” of endostatin can be, for example, such as 30% or higher, 50% or higher, 60% or higher, 70% or higher, 80% or higher or 90% or higher of those of a natural endostatin, for example, a natural human endostatin. The “functional variant” may be a naturally occurring mutant or an artificial mutant, such as a mutant obtained by site-directed mutagenesis, or a mutant produced by a genetic recombination method.

The biological activities of the “functional variant” can be detected by methods well known in the art for detecting endostatin activities. For example, HMEC cells can be selected, and the inhibition rate of HMEC cell migration by a functional variant is analyzed using the Migration (Tranwell Assay) method, and the number of cells is counted to reflect the protein activity (see Luo yongzhang et al., Endostatin inhibits tumourlymphangiogenesis and lymphatic metastasis via cell surface nucleolin on lymphangiogenic endothelial cells (J Pathol 2010; 222: 249-260)).

In some embodiments of the present invention, the “endostatin” may be a functional variant of natural human endostatin, such as ZBP-Endostatin (under Trade Name of Endostar), which is an ES variant obtained by adding 9 additional amino acids (MGGSHHHHH) to the N-terminus of natural human ES, in order to increase soluble expression and facilitate purification. The amino acid sequence of ZBP-Endostatin is:

(SEQ ID NO: 3)
MGGSHHHHHHSHRDFQPVLHLVALNSPLSGGMRGIRGADFQCFQQARAVG
LAGTFRAFLSSRLQDLYSIVRRADRAAVPIVNLKDELLFPSWEALFSGSE
GPLKPGARIFSFDGKDVLRHPTWPQKSVWHGSDPNGRRLTESYCETWRTE
APSATGQASSLLGGRLLGQSAASCHHAYIVLCIENSFMTASK;

wherein the Met at its N-terminus is sometimes partially deleted when it is expressed by E. coli.

Other examples of endostatin functional variants include those endostatin mutants disclosed in the PCT International Application PCT/CN2012/081210, such as ES006, ES008, ES011, S02, S09, Z006, Z008, ZN1, etc. (all of which are incorporated herein by reference in their entirety).

The “endostatin” as described herein may also be a derivative or a modified product (such as a polyethylene glycol modified product) of natural endostatin or a functional variant thereof, which has substantially the same biological functions as natural endostatin, such as the activities of inhibiting the proliferation, migration and in vivo angiogenesis of vascular endothelial cells.

In some embodiments of the present invention, the “endostatin” may be a derivative of natural human endostatin, for example, a product resulted from the modification of a natural human ES with a monomethoxypolyethylene glycol propionaldehyde (mPEG-ALD) with a molecular weight of 20 kDa, wherein the coupling site is the activated mPEG-ALD aldehyde group and the N-terminal α-amino.

The “DNA-dependent protein kinase catalytic subunit (DNA-PKcs)” as described herein is a catalytic subunit of the DNA-PK complex, is a member of the phosphatidylinositol-3-kinase-like kinase family (PIKK) and plays a central role in nonhomologous end joining (NHEJ) repair.

The “treatment regimen for inducing DNA double-strand break” as described herein can be any form of treatment regimen as long as the treatment regimen can finally result in DNA double-strand break. The treatment regimen may be, for example, a treatment regime that directly causes a double-strand break in DNA. The treatment regimen may also be, for example, a treatment regimen in which a single-strand break in DNA occurs, thereby finally leading to a double-strand break in DNA. The treatment regimen may also be, for example, a treatment regimen that causes chromosomal damage, thereby finally leading to DNA double-strand break. The treatment regimen may also be a treatment regimen that causes DNA double-strand break, for example by DNA-PKcs inhibition. The treatment regimen may be radiotherapy, or a chemotherapeutic agent, may be one chemotherapeutic agent or a combination of more different chemotherapeutic agents, or may be a combination treatment regimen of one or more chemotherapeutic agents and radiotherapy.

Radiotherapy regimens for inducing DNA double-strand break are well known in the art. Radiotherapy can be performed by any suitable technique, including but not limited to electromagnetic radiation or ionizing radiation. The electromagnetic radiation may be, for example, X-rays, gamma rays; and the ionizing radiation may be, for example, an electron beam or the like. Dosimetry for radiotherapy is also well known in the art.

The term “chemotherapeutic agent” as used herein refers to a compound that has a therapeutic effect on tumor or cancer. The chemotherapeutic agent that can be used in the present invention is a chemotherapeutic agent that can induce DNA double-strand break. For example, it may be a topoisomerase II inhibitor, an alkylating agent, a platinum complex, bleomycin, or doxorubicin, such as, but not limited to, Etoposide or Doxorubicin.

In the present invention, an endostatin can be used in combination with a treatment regimen for inducing DNA double-strand break, for treating tumor or cancer. The tumor or cancer is a tumor or cancer suitable for treatment with a treatment regime for inducing DNA double-strand break.

The “p53” as described herein is a tumor suppressor gene that encodes a protein with a molecular weight of 53 kDa, called P53. The biological function of normal P53 is similar to that of the “guardian of the genome”, which checks for DNA damage site at G1 phase and monitors genome integrity. If there is damage, the P53 protein prevents DNA replication to provide enough time for the damaged DNA to be repaired. If the repair fails, the P53 protein will trigger apoptosis. If two copies of the p53 gene are mutated, which causes the P53 protein to lose its normal function, the proliferation of cells will be out of control and the cells will become cancerous.

The tumor or cancer that can be treated using the agent or treatment regimen described herein may be deficient in P53 function. The tumor or cancer with loss of P53 function include tumor or cancer with loss of P53 function in some or all of the cells. The cells with loss of P53 function may be cells with complete loss of P53 function or cells with partial loss of P53 function. The complete loss of P53 function may be, for example, complete loss of the biological function of P53 compared to a cell in which P53 is wild-type. The partial loss of P53 function may be, for example, partial loss or attenuation of the biological function of P53 compared to a cell in which P53 is wild-type. A cell with a loss of P53 function may have, for example, a decreased or disrupted expression and/or activity of P53 protein compared to a normal cell, which can be caused, for example, by a mutation in its coding nucleic acid, or by a mutation in the nucleic acid encoding its regulator. The decreased or disrupted expression and/or activity of P53 protein can be manifested as absence of P53 protein, complete inactivation of P53 protein, decrease in the amount of P53 protein, or decrease in the function of P53 protein. The “mutation” may include insertion, deletion or substitution of one or more nucleotides.

In cells with complete loss of P53 function, they are not sensitive to radiotherapy or chemotherapeutic agents. This is because although radiotherapy or chemotherapy agents cause DNA damage in cells, which activates the DNA-PKcs signaling pathway mediates DNA damage repair through non-homologous end joining pathways, due to the loss of P53 function, Caspase is not easily activated. In the presence of DNA-PKcs, damage can be repaired in time, which makes the cells resistant to the treatment regimen to some extent and does not lead to apoptosis easily. Endostatin can inhibit the activity of DNA-PKcs, promote the accumulation of DNA damage in cells, and promote apoptosis. Therefore, for cells with loss of function of P53, especially tumor or cancer cells, which are treated with a treatment regimen for inducing DNA double-strand break such as radiotherapy or a chemotherapy agent, endostatin can increase the sensitivity of these cells to the treatment regimen for inducing DNA double-strand break, and promote the therapeutic effects of the treatment regimen for inducing DNA double-strand break.

For cells without loss of P53 function (that is, P53 functions normally), the cells are more sensitive to radiotherapy or a chemotherapy agent. In the early stage of apoptosis, DNA-PKcs will be degraded by Caspase-3 activated by the P53 pathway, and endostatin does not significantly increase its sensitivity. However, since endostatin can inhibit DNA-PKcs activity, the use of endostatin can reduce the dose of a treatment regimen for inducing DNA double-strand break such as radiotherapy or a chemotherapeutic agent. The “reduce the dose of a treatment regimen for inducing DNA double-strand break” refers to that when compared with the dose of a treatment regimen for inducing DNA double-strand break used alone, the dose of the treatment regimen for inducing DNA double-strand break can be smaller when used in combination with endostatin, but can also achieve the same therapeutic effect. In other words, when the treatment regimen for inducing DNA double-strand break is used in combination with endostatin, its dose applied does not exceed the dose usually applied when the treatment regimen for inducing DNA double-strand break is used alone.

When the treatment regimen for inducing DNA double-strand break is radiotherapy, the dose refers to a radiation dose. The dose usually applied when the radiotherapy is performed alone may be, for example, 1-100 Gy. When the radiotherapy is used in combination with endostatin, the radiation dose can be reduced by 1%, 5%, 10%, 20%, 30%, 40%, 50%, 60%, 70%, 80%, or even 90% compared with the one when the radiotherapy is applied alone.

When the treatment regimen for inducing DNA double-strand break is a chemotherapeutic agent, the dose refers to an administration dose of the chemotherapeutic agent. When a chemotherapeutic agent is used in combination with endostatin, the dose of the chemotherapeutic agent can be reduced by 1%, 5%, 10%, 20%, 30%, 40%, 50%, 60%, 70%, 80%, or even 90% compared with the one when the chemotherapy agent is administrated alone.

For cells with partial loss of P53 function, which are treated with a treatment regimne for inducing DNA double-strand break, vascular endothelial inhibition can have both of the above effects, which can not only increase the sensitivity of these cells to the treatment regimen for inducing DNA double-strand break and promote the therapeutic effect of the treatment regimen for inducing DNA double-strand break, but also reduce the dose of the treatment regimen for inducing DNA double-strand break.

The amount of endostatin in the pharmaceutical composition, combination drugs or kit of the present invention, or the amount of endostatin in the use or method of the present invention, is an amount capable of effectively inhibiting DNA-PKcs in tumor cells.

The tumors described herein include benign and malignant tumors, and doubling of tumor cells refers to uncontrolled and progressive histological cell growth. Some of these growths are benign, but others are “malignant” and can cause the death of organism. In addition to exhibition of aggressive cell proliferation, malignant tumors can infiltrate surrounding tissues and metastasize. The cancers described herein generally include malignant solid tumors, as well as leukemia, lymphoma, and other cancers that do not usually exist as tumor masses but are distributed in the blood vessels or lymphoreticular system.

The treatment of tumors or cancers by using the regimen of the present invention is achieved by inducing apoptosis. Therefore, the therapeutic effect of the regimen of the present invention on tumor or cancer is not closely related to the origin of tumor cells or cancer cells, but is related to the molecular typing of cells. For example, tumors or cancers suitable for treatment by the method of the present invention may be P53-deficient. Tumors or cancers that can be treated with the regimen of the present invention include, but are not limited to, solid tumors/malignant tumors, myxoid and round cell carcinomas, locally advanced tumors, metastatic cancers, human soft tissue sarcomas (including Ewing's sarcoma), cancer metastases (including lymphatic metastases), squamous cell carcinoma (especially head and neck squamous cell carcinoma, esophageal squamous cell carcinoma), oral cancer, hematological malignancies (including multiple myeloma), leukemia (including acute lymphocytic leukemia, acute nonlymphocytic leukemia, chronic lymphocytic leukemia, chronic myeloid leukemia, and hairy cell leukemia), exudative lymphoma (body cavity-based lymphoma), thymic lymphoma, lung cancer (including small cell cancer), skin T-cell lymphoma, Hodgkin's lymphoma, non-Hodgkin's lymphoma, adrenocortical cancer, ACTH-producing tumor, non-small cell lung cancer, breast cancer (including small cell and ductal cancer), gastrointestinal cancer (including gastric cancer, colon cancer, colorectal cancer), polyps associated with colorectal tumor formation, pancreatic cancer, liver cancer, urinary cancer (including bladder cancer, including primary superficial bladder tumors, invasive transitional cell carcinoma of bladder and muscular invasive bladder cancer), prostate cancer, malignant tumors of female reproductive tract (including ovarian cancer, primary peritoneal epithelial cell tumor, cervical cancer, endometrial cancer, vaginal cancer, vulvar cancer, uterine cancer, and follicular solid cancer), malignant tumors of male reproductive tract (including testicular and penile cancer), kidney cancer (including renal cell carcinoma), brain cancer (including endogenous brain tumor, neuroblastoma, astrocytic brain tumor, glioma, metastatic tumor cell infiltration in the central nervous system), bone cancer (including osteoma and osteosarcoma), skin cancer (including melanoma, progressive tumors of human skin keratinocytes, squamous cell carcinoma), thyroid cancer, retinoblastoma, neuroblastoma, peritoneal leakage, malignant pleural leakage, mesothelioma, Wilms tumor, gallbladder cancer, trophoblastoma, hemangiopericytoma, and Kaposi's sarcoma. In some embodiments, the regimen of the present invention is suitable for treating non-small cell lung cancer or melanoma.

The “treatment” or “therapeutic effect” described in the present invention includes, for example, inhibiting or reducing the growth, infiltration or metastasis of tumor or cancer cells, etc., or reducing symptoms, prolonging the survival period, etc.

The subjects described herein may include, but are not limited to, vertebrates, mammals, rodents (e.g. hamsters, rats, mice), canines (e.g. dogs), felines (e.g. cats), horses, pigs, cattle, sheep, goats, primates (e.g. chimpanzees, apes or monkeys), or humans. The cells described herein may include, but are not limited to, cells of vertebrates, mammals, rodents (e.g. hamsters, rats, mice), canines (e.g. dogs), felines (e.g. cats), horses, pigs, cattle, sheep, goats, primates (e.g. chimpanzees, apes or monkeys), or humans.

In the present invention, the administration of endostatin and a treatment regimen for inducing DNA double-strand break can be performed simultaneously or sequentially. When performed sequentially, endostatin can be administered first, followed by a treatment regimen for inducing DNA double-strand break, or a treatment regimen for inducing DNA double-strand break can be performed first, followed by administration of endostatin.

The present invention also provides a method of inhibiting DNA-PKcs activity in a biological sample, comprising contacting the biological sample with endostatin. The “biological sample” described herein refers to a sample in vitro of a living organism, including but not limited to a cell culture or an extract thereof; a biopsy material obtained from a mammal or an extract thereof; and blood, saliva, urine, feces, semen, tears, or other body fluids or their extracts. Inhibition of DNA-PKcs activity in a biological sample can be used for a variety of purposes known to those skilled in the art. Examples include, but are not limited to, inhibition of DNA-PKcs in a bioassay. In one embodiment, the method of inhibiting DNA-PKcs activity in a biological sample is limited to a non-therapeutic method.

The term “pharmaceutically acceptable carrier” as used herein refers to substances that can be safely administered, such as solid or liquid diluents, fillers, antioxidants, and stabilizers. Depending on the route of administration, a variety of different carriers well known in the art can be administered, including, but not limited to, sugars, starches, celluloses and derivatives thereof, maltose, gelatin, talc, calcium sulfate, vegetable oils, synthetic oils, polyols, alginic acid, phosphate buffers, emulsifiers, isotonic saline, and/or pyrogen-free water.

As used herein, the term “combination of drugs” or “combination” includes various specific combinations of various drugs or treatment regimens that are used in combination. For example, when different drugs are used in combination, the various drugs used in combination may exist in the form of a mixture or a complex, or may exist in separate entities, respectively, and be administered to a subject simultaneously or sequentially. When a drug and a non-drug treatment regimen are used in combination, the drug and the treatment regimen can be administered to the subject simultaneously or sequentially.

The pharmaceutical composition, endostatin, and/or treatment regimen for inducing DNA double-strand break described herein can be administered in a therapeutically effective amount. The term “therapeutically effective amount” as used herein refers to an amount of an active compound sufficient to cause a biological or medical response desired by a clinician in a subject. In the present invention, the therapeutically effective amount of endostatin may be, for example, an amount capable of effectively inhibiting DNA-PKcs in tumor cells. In the present invention, the “therapeutically effective amount” of the pharmaceutical composition, endostatin, and/or treatment regimen for inducing DNA double-strand break can be determined by those skilled in the art according to factors such as the route of administration, the body weight, age, and condition of a subject. For example, for a pharmaceutical composition, endostatin and/or a chemotherapeutic agent, a typical daily dose may range from 0.01 mg to 100 mg of an active ingredient per kg of body weight, and for radiotherapy, a typical daily dose can range from 1 to 5 Gy.

The drug provided by the present invention can be formulated into a clinically acceptable dosage form such as powder and injection. The subject may be administered with the pharmaceutical composition, endostatin, and/or chemotherapeutic agent of the present invention by any suitable route, for example, oral administration, intravenous infusion, intramuscular injection, subcutaneous injection, subperitoneal administration, rectal administration, sublingual administration, or inhalation, transdermal administration.

The various methods mentioned in the present invention can be performed in vivo or in vitro.

The various methods mentioned in the present invention may be non-therapeutic.

Unless otherwise stated, scientific and technical terms used in this specification shall have the meanings commonly understood by those of ordinary skill in the art. In general, nomenclatures and techniques associated with cell and tissue culture, molecular biology, immunology, microbiology, genetics, and protein and nucleic acid chemistry as used in this specification are well known and commonly used in the art.

Unless otherwise stated, the methods and techniques used in this specification are generally performed according to the well known and routine methods in the art and in the manner described in the various references set forth or cited in this specification.

### EXAMPLES

In the following examples, the endostatin used is a recombinant natural human endostatin expressed by E. coli, which is from Beijing Protgen Biotechnology Development Co., Ltd. (Protgen) and has the following amino acid sequence:

(SEQ ID NO: 2)
MHSHRDFQPVLHLVALNSPLSGGMRGIRGADFQCFQQARAVGLAGTFRAF
LSSRLQDLYSIVRRADRAAVPIVNLKDELLFPSWEALFSGSEGPLKPGAR
IFSFDGKVDLRHPTWPQKSVWHGSDPNGRRLTESYCETWRTEAPSATGQA
SSLLGGRLLGQSAASCHHAYIVLCIENSFMTASK;

wherein the Met at the N-terminus is sometimes partially deleted when expressed by E. coli.

The ZBP-Endostatin used is an endostatin variant obtained by adding 9 additional amino acids (MGGSHHHHH) to the N-terminus of the natural human endostatin (Fu Y et al. Biochemistry 2010, 49, 6420-6429), provided by Beijing Protgen Biotechnology Development Co., Ltd. (Protgen), and the amino acid sequence is as follows:

(SEQ ID NO: 3)
MGGSHHHHHHSHRDFQPVLHLVALNSPLSGGMRGIRGADFQCFQQARAVG
LAGTFRAFLSSRLQDLYSIVRRADRAAVPIVNLKDELLFPSWEALFSGSE
GPLKPGARIFSFDGKDVLRHPTWPQKSVWHGSDPNGRRLTESYCETWRTE
APSATGQASSLLGGRLLGQSAASCHHAYIVLCIENSFMTASK;

wherein the Met at the N-terminus is sometimes partially deleted when expressed by E. coli.

The PEG-Endostatin used is a polyethylene glycol (PEG) modified natural human endostatin, which is a product obtained by modifyingthe natural human endostatin molecule with a monomethoxypolyethylene glycol propionaldehyde (mPEG-ALD) with a molecular weight of 20 kDa, wherein the coupling site is the activated mPEG-ALD aldehyde group and N-terminal α-amino group of Endostatin. It was provided by Beijing Protgen Biotechnology Development Co., Ltd. (Protgen).

Non-small cell lung cancer H1299 (P53 deficient), A549 (P53 wild type) cell line, and melanoma MDA-MB-435S (P53 mutant) and A375 (P53 wild type) cell line were purchased from the National Experimental Cell Resource Sharing Platform (China Infrastructure of Cell Line Resource). The P53 stably overexpressing H1299 cell line (H1299-P53) and the P53 stably knockdown A549 cell line (A549-NC) were constructed using a lentivirus kit (purchased from Shanghai Genepharma), wherein the P53 sequence targeted by the shRNA used to knock down P53 in A549 cells was 5′-GACTCCAGTGGTAATCTAC-3′ (SEQ ID NO: 4). Virus transfection and the construction of stable cell lines were performed according to the kit instructions. H1299-NC and A549-NC were stable cell lines obtained by transfecting H1299 cells and A549 cells with blank lentivirus, respectively.

### Example 1

### Endostatin Interacted Directly with DNA-PKcs

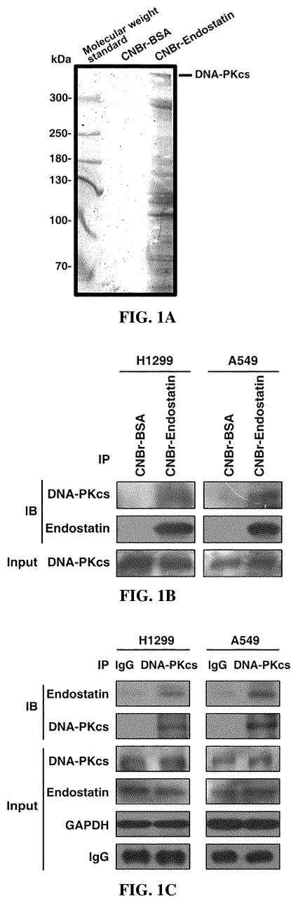

CNBr-activated Sepharose 4B used in this example was purchased from Sigma. First, according to the instructions, endostatin and bovine serum albumin (BSA) were coupled to CNBr-activated Sepharose 4B, respectively, and incubated with A549 cell lysate. The pull down proteins were subjected to SDS-PAGE electrophoresis and stained with Coomassie blue. By Mass spectrometric analysis, DNA-PKcs was identified as a protein interacted with Endostatin (FIG. 1A). Then, immunoblotting experiments demonstrated that the DNA-PKcs protein in both H1299 and A549 cell lysates can be precipitated by CNBr-activated Sepharose 4B coupled with Endostatin (FIG. 1B). To further verify the interaction between the two, H1299 and A549 cells were incubated with Endostatin for 2 hours for endocytosis. The cell lysate was mixed with the antibody of DNA-PKcs, and then co-incubated with protein A/G beads. The immunoblotting assay verified that Endostatin could be co-precipitated by DNA-PKcs, which proved the interaction between the two (FIG. 1C).

### Example 2

### Endostatin Inhibited the Activation of DNA-PKcs Signaling Pathway in Tumor Cells Induced by a Chemotherapeutic Agent

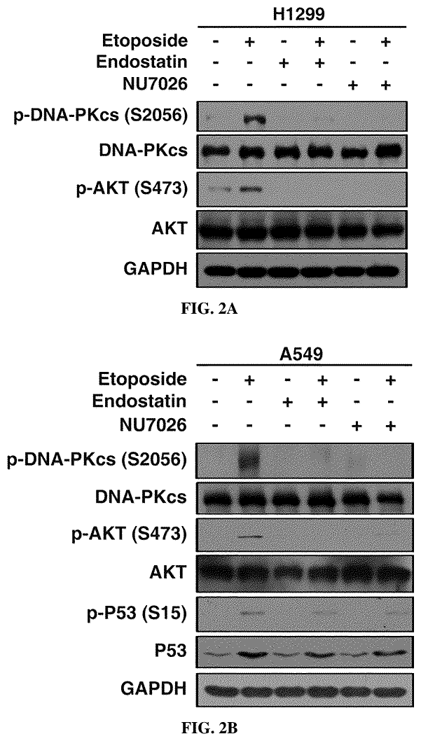

In this example, the known substrates of DNA-PKcs, AKT and P53, were used as detection indicators to verify the inhibitory effect of Endostatin on the DNA-PKcs pathway. Tumor cells (including non-small cell lung cancer H1299 and A549 cells) were divided into six treatment groups. The first group: negative control; the second group: Etoposide (10 μM, 1 h) treatment; the third group: Endostatin (10 μg/ml, 1 h) treatment; the fourth group: combined treatment with Endostatin (10 μg/ml, added 1 h before Etoposide) and Etoposide (10 μM, 1 h); the fifth group: NU7026 (10 μM, 1 h) treatment; the sixth group: combined treatment with NU7026 (10 μM, added 1 h before Etoposide) and Etoposide (10 μM, 1 h), wherein, NU7026 was a DNA-PKcs inhibitor. The immunoblotting assay results showed (FIG. 2A-2B, wherein p-DNA-PKcs (S2056) represents the phosphorylation level of DNA-PKcs, S2056 represents its phosphorylation site; wherein p-AKT (S473) represents the phosphorylation level of AKT, and S473 represents its phosphorylation site; wherein p-P53 (S15) represents the phosphorylation level of P53, and S15 represents its phosphorylation site), compared with the control group, Etoposide treatment activated the level of the DNA-PKcs signaling pathway; Endostatin treatment did not affect the level of the DNA-PKcs signaling pathway; the combination of Endostatin and Etoposide inhibited activation of the DNA-PKcs signaling pathway by Etoposide. NU7026 treatment did not affect the level of the DNA-PKcs signaling pathway; the combination of NU7026 and Etoposide inhibited activation of the DNA-PKcs signaling pathway by Etoposide.

### Example 3

### Endostatin Delayed DNA-PKcs-Mediated Repair of DNA Damage

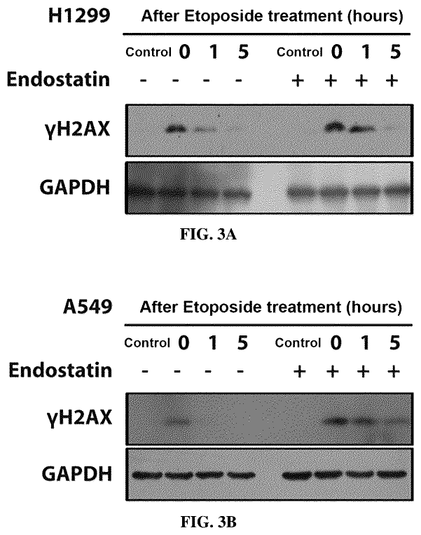

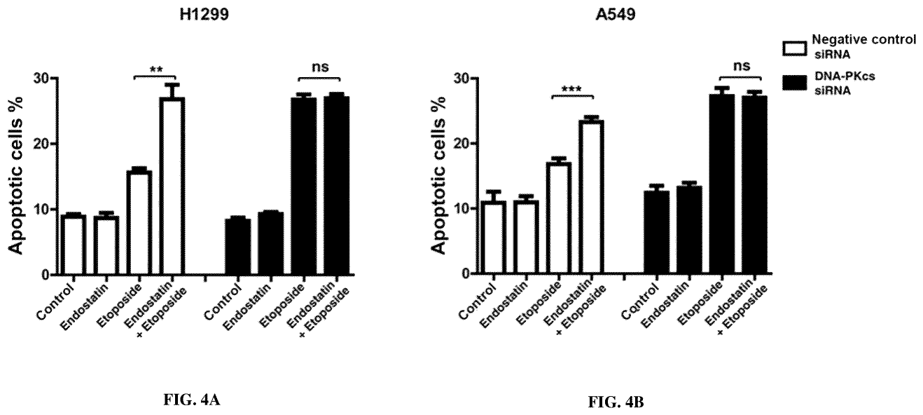

In this example, the level of γH2AX was used to reflect the degree of DSBs damage. Tumor cells (including H1299 and A549 cells) were divided into eight treatment groups. The first group: negative control, cells were collected at 0 hour; the second group: cells were collected after Etoposide (10 μM) treatment for 1 hour; the third group: after Etoposide (10 μM) treatment for 1 hour, it was replaced with fresh medium, and cells were collected after 2 hours; the fourth group, aftet Etoposide (10 μM) treatment for 1 hour, it was replaced with fresh medium, and cells were collected after 5 h; the fifth to eighth groups were first pretreated with Endostatin (10 μg/ml) for 1 h, and the subsequent treatments corresponded to those of the first to fourth groups, respectively. The immunoblotting assay results showed that the DNA damage produced by Etoposide-treated cells was gradually repaired after the drug was removed from the medium in the first to fourth groups, and the γH2AX level returned to baseline after 5 hours. In the fifth to eighth group, the use of Endostatin in combination delayed the reduction of γH2AX level, which proved that the repair of DSBs damage in cells was delayed (FIG. 3A-3B).

### Example 4

### Endostatin Regulated the Sensitivity of Tumor Cells to Chemotherapy Depending on the Presence of DNA-PKcs

In this example, tumor cells (including H1299 and A549 cells) were divided into eight treatment groups. In the first four groups, cells were transfected with control siRNA, and in the last four groups, cells were transfected with siRNA that specifically knocked down DNA-PKcs. The siRNA sequence of DNA-PKcs is: forward 5′-CAGGGUUUAAUCAGAAUAUTT-3′ (SEQ ID NO: 5), reverse 5′-AUAUUCUGAUUAAACCCUGTT-3′ (SEQ ID NO: 6). The siRNA transfection method was performed according to the instructions of the transfection reagent Lipofectamine 2000. Then, in the eight groups, the cells were treated with drugs. Groups 1 and 4: control group; Groups 2 and 5: Endostatin (10 μg/ml, 16 h) treatment; Groups 3 and 6: Etoposide (10 μM for H1299, 1 μM for A549, 16 h) treatment; Groups 4 and 8: combined treatment with Endostatin (10 μg/ml, added 1 h before Etoposide) and Etoposide (10 μM for H1299, 1 μM for A549, 16 h). Cells were collected and operations were performed according to the instructions of Annexin V-FITC/PI Apoptosis Detection Kit. Apoptosis was detected by flow cytometry. A549 cells were P53 wild-type cells, and H1299 cells were P53-deficient cells. The results showed that A549 cells were more sensitive to the chemotherapeutic agent, and when the same degree of apoptosis was caused, the Etoposide concentration in H1299 cells was much higher than that in A549 cells. On the basis of this, the addition Endostatin would further promote Etoposide-induced apoptosis (FIG. 4A-4B). After knocking down DNA-PKcs, the role of Endostatin in promoting apoptosis induced by Etoposide was weakened.

### Example 5

### Endostatin Synergized with Etoposide Inhibited the Activity of P53-Deficient Tumor Cells

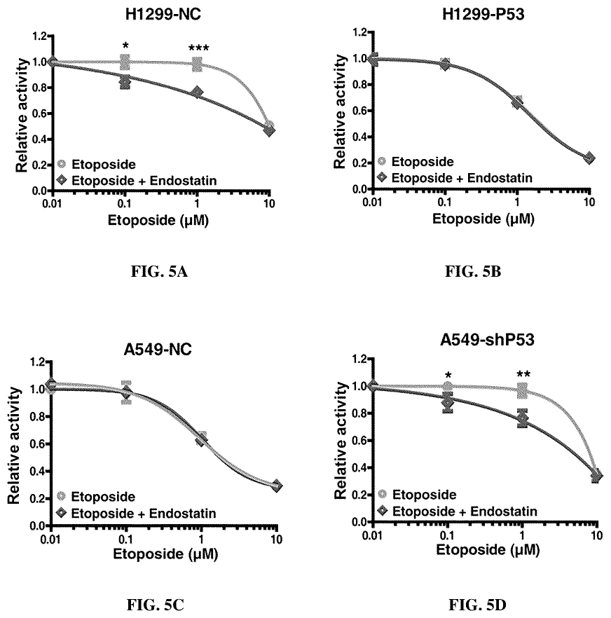

First, a lentivirus kit (purchased from Shanghai Genepharma) was used to construct a P53 stably overexpressing H1299 cell line (H1299-P53) and a P53 stably knockdown A549 cell line (A549-NC). The P53 sequence targeted by the shRNA used to knock down P53 in A549 cells was 5′-GACTCCAGTGGTAATCTAC-3′ (SEQ ID NO: 4). Virus transfection and the construction of stable cell lines were performed according to the kit instructions. The constructed tumor cells (H1299-NC, H1299-P53, A549-NC, A549-shP53) were seeded into 96-well plates (1000 cells per well) and divided into eight treatment groups. In the first group to the fourth group, a medium containing different concentrations of Etoposide (0 μM, 0.1 μM, 1 μM, 10 μM) was added to each well. The fifth to eighth groups were first pretreated with a medium containing 10 μg/ml Endostatin for 1 h, and then a medium containing different Etoposide concentrations was added so that the final concentrations of Etoposide in the fifth to eighth groups were 0 μM, 0.1 μM, 1 μM, and 10 μM, respectively. After treatment for 12 hours, the drug-containing medium was replaced with a normal medium and the cells were further cultured for 72 hours. Cell activity was measured according to the instructions of the CCK8 kit (Dojindo, Tokyo, Japan). The results showed that for P53-deficient tumor cells (FIGS. 5A and 5D), Endostatin could enhance the toxicity of Etoposide to tumor cells. For tumor cells with normal P53 function (FIGS. 5B and 5C), Etoposide could significantly inhibit the activity of tumor cells, and Endostatin did not significantly enhance the inhibitory effect of Etoposide.

### Example 6

### Endostatin Synergized with Etoposide Inhibited the Survival of P53-Deficient Tumor Cells

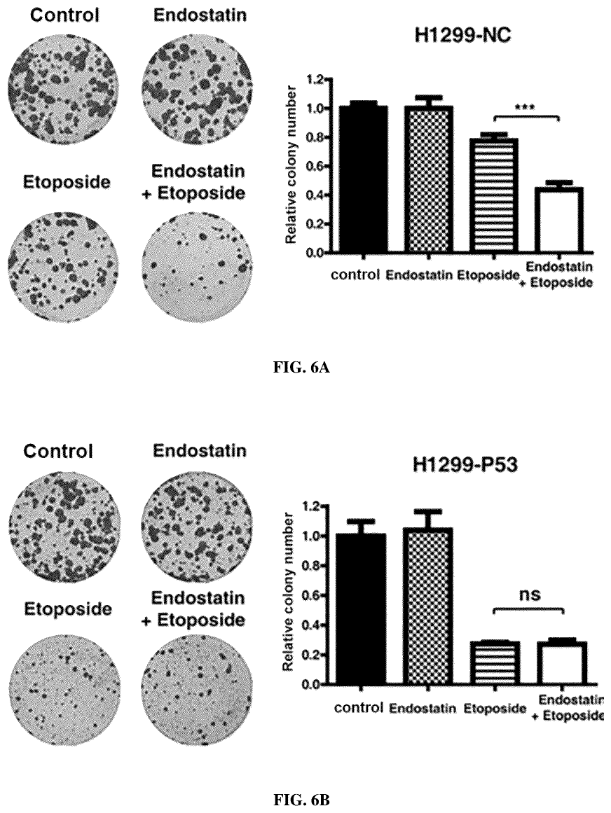

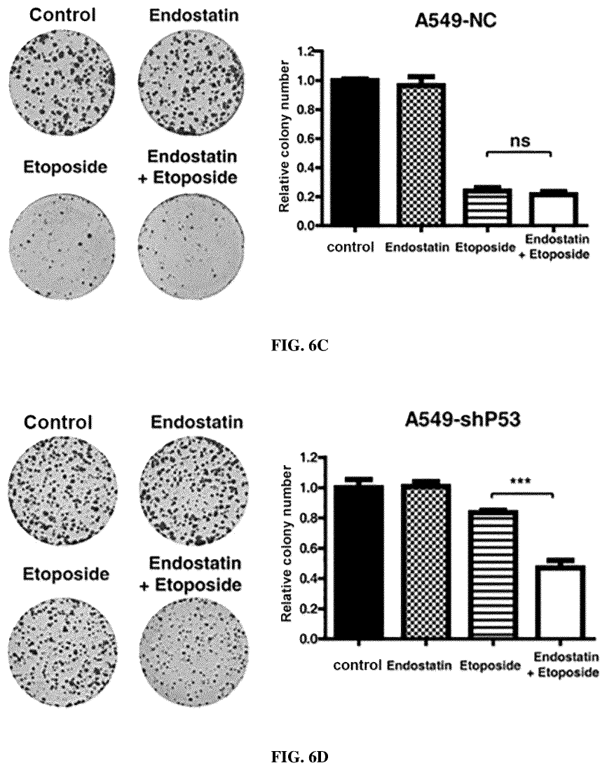

Tumor cells (H1299-NC, H1299-P53, A549-NC, A549-shP53) were divided into four treatment groups. The first group: negative control; the second group: Endostatin (10 μg/ml, 16 h) treatment; the third group: Etoposide (1 μM, 16 h) treatment; the fourth group: combined treatment with Endostatin (10 μg/ml, added 1 h before Etoposide) and Etoposide (1 μM, 16 h). After the treatment, the cells were digested with trypsin, counted, and re-plated in a six-well plate with 1000 cells per well. The cells were cultured in a normal medium for 14 days to form cell colonies. The results of cell colony counting showed that for P53-deficient tumor cells (FIGS. 6A and 6D), Endostatin could enhance the inhibitory effect of Etoposide on tumor cell colony-forming ability and reduce the viability of tumor cells. For tumor cells with normal P53 function (FIGS. 6B and 6C), Etoposide could significantly inhibit the colony-forming ability of tumor cells, and Endostatin did not significantly enhance theinhibitory effect of Etoposide.

### Example 7

### Endostatin Synergized with Etoposide Promoted the Apoptosis of P53-Deficient Tumor Cells

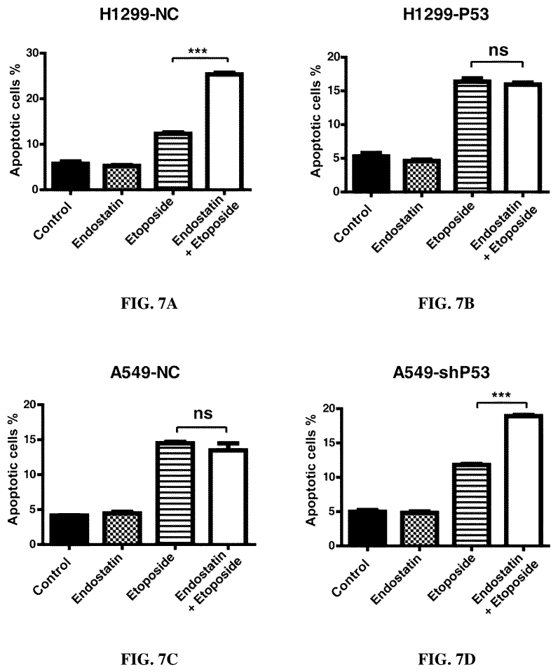

Tumor cells (H1299-NC, H1299-P53, A549-NC, A549-shP53) were divided into four treatment groups. The first group: negative control; the second group: Endostatin (10 μg/ml, 16 h) treatment; the third group: Etoposide (1 μM, 16 h) treatment; the fourth group: combined treatment with Endostatin (10 μg/ml, added 1 h before Etoposide) and Etoposide (1 μM, 16 h). The apoptotic cells and adherent cells floating in the culture medium were collected, and the cells were suspended in 500 μl PBS containing 0.05 mg/ml propidium iodide (PI), stained for 15 min at room temperature in dark, and detected by flow cytometry (BD FACS Calibur). PI-positive cells represent apoptotic cells. The results showed that for P53-deficient tumor cells (FIGS. 7A and 7D), Endostatin could enhance the ability of Etoposide to promote the apoptosis of tumor cells. For tumor cells with normal P53 function (FIGS. 7B and 7C), Etoposide could significantly promote the apoptosis of tumor cells, and Endostatin did not significantly enhance the pro-apoptotic effect of Etoposide.

### Example 8

### Endostatin Synergized Doxorubicin Inhibited the Activity of P53-Deficient Tumor Cells

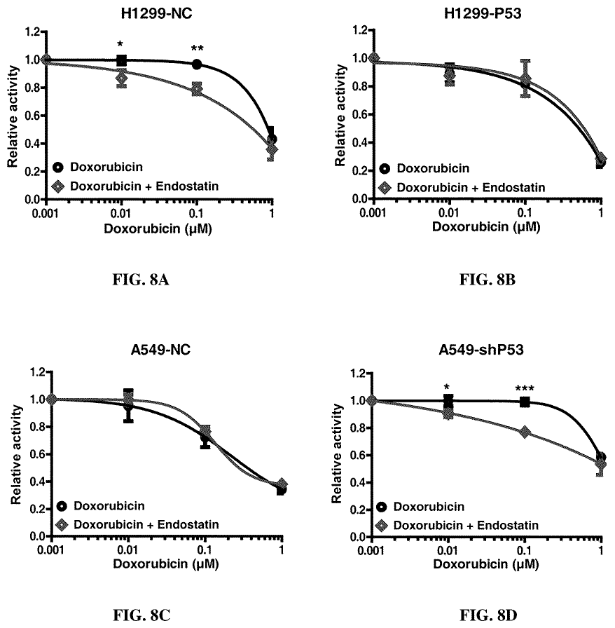

In this example, another chemical drug, Doxorubicin, was used to verify that Endostatin can enhance the sensitivity of P53-deficient tumor cells to chemotherapy. Operation process was the same as in Example 5. The results showed that for P53-deficient tumor cells (H1299-NC and A549-shP53) (FIGS. 8A and 8D), Endostatin could enhance the toxicity of Doxorubicin to tumor cells. For tumor cells with normal P53 function (H1299-P53 and A549-NC) (FIGS. 8B and 8C), Doxorubicin could significantly inhibit the activity of tumor cells, and Endostatin did not significantly enhance the inhibitory effect of Doxorubicin.

### Example 9

### The Inhibitory Effect of Etoposide Synergized with Endostatin was Applicable to Other Types of Tumor Cells

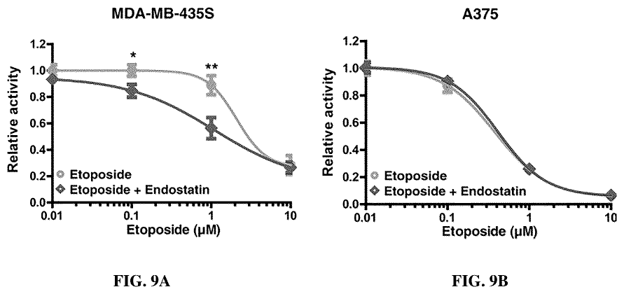

This example used two cell lines of another tumor type (melanoma), the MDA-MB-435S (P53 mutant) and A375 (P53 wild type) cell lines, to verify the regulatory function of Endostatin on the chemotherapeutic sensitivity of other types of tumor cells with different P53 status. Operation process was the same as in Example 5. The results showed that for MDA-MB-435S cells (FIGS. 9A and 9D), Endostatin could enhance the toxicity of Etoposide to tumor cells. For A375 cells (FIGS. 9B and 9C), Etoposide could significantly inhibit the activity of tumor cells, and Endostatin did not significantly enhance the inhibitory effect of Etoposide.

### Example 10

### Endostatin Inhibited DNA-PKcs to Repair Cell Damage, Induced Activation of Apoptotic Pathway and Degradation of DNA-PKcs in P53-Deficient Tumor Cells

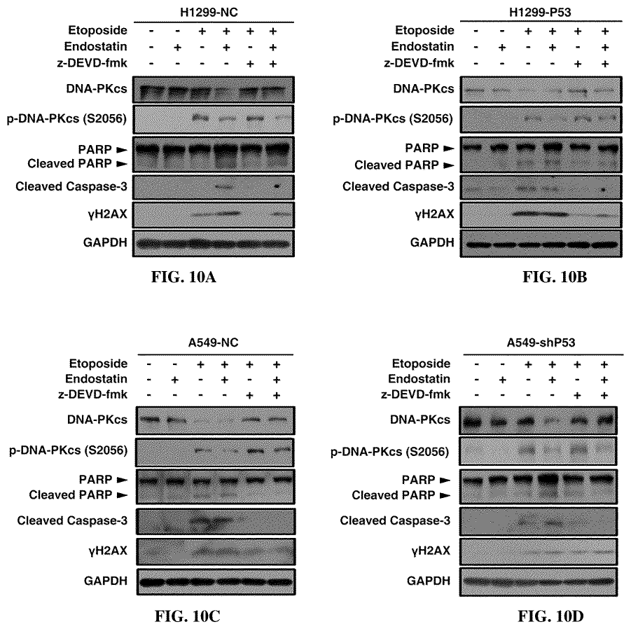

Studies have shown that DNA-PKcs is a substrate of Caspase-3, and activated Caspase-3 will cleave DNA-PKcs to degrade it (T. Itoh et al. Journal of dermatological science, 2001; 25, 72-77, K. M. Henkels et al. Cancer research, 1997; 57, 4488-4492). In this example, tumor cells (H1299-NC, H1299-P53, A549-NC, A549-shP53) were divided into six treatment groups. The first group: negative control; the second group: Endostatin (10 μg/ml, 16 h) treatment; the third group: Etoposide (10 μM, 16 h) treatment; the fourth group: combined treatment with Endostatin (10 μg/ml, added 1 h before Etoposide) and Etoposide (10 μM, 16 h); the fifth group: combined treamtment with z-DEVD-fmk (10 μM, added 1 h before Etoposide) and Etoposide (10 μM, 16 h); the sixth group: combined treatment with z-DEVD-fmk (10 μM) and Endostatin (10 μg/ml) added 1 h before Etoposide, and then Etoposide (10 μM, 16 h), wherein, z-DEVD-fmk was a caspase-3 inhibitor. Immunoblotting assay detected DNA-PKcs, PARP, γH2AX and Caspase-3 signaling pathways. The results (as shown in FIG. 10A-10D, in which p-DNA-PKcs (S2056) represents the phosphorylation level of DNA-PKcs, and S2056 represents its phosphorylation site) showed that compared with the control group, in both P53-deficient tumor cells (FIGS. 10A and 10D) and tumor cells with normal P53 function (FIGS. 10B and 10C), Endostatin did not affect the above-mentioned signaling pathway levels; for P53-deficient tumor cells, Etoposide had no significant effect on the above signaling pathways; as compared with Etoposide, the combination of Endostatin and Etoposide significantly promoted the activation of Caspase-3, and degradation of DNA-PKcs and PARP, with a significant increase in γH2AX; the combination of z-DEVD-fmk and Etoposide had no significant effect on the above signal levels; z-DEVD-fmk could reverse the synergistic effect of Endostatin and Etoposide to some extent. For tumor cells with normal P53 function, both the treatment with Etoposide alone and the combined treatment with Endostatin and Etoposide showed significant activation of Caspase-3, degradation of DNA-PKcs and PARP, and an increase in γH2AX; the combination of z-DEVD-fmk and Etoposide reversed the effect of Etoposide to some extent; the combination of z-DEVD-fmk, Endostatin and Etoposide showed that z-DEVD-fmk reversed the effect of Etoposide, the protein level of DNA-PKcs was restored, and the synergistic effect of Endostatin and Etoposide was reflected to a certain extent.

### Example 11

### Endostatin Synergized with Etoposide Up-Regulated the Expression of Pro-Apoptotic Molecules in P53-Deficient Tumor Cells

Tumor cells (H1299-NC, H1299-P53, A549-NC, A549-shP53) were divided into four treatment groups. The first group: negative control; the second group: Endostatin (10 μg/ml, 16 h) treatment; the third group: Etoposide (1 μM, 16 h) treatment; the fourth group: combined treatment with Endostatin (10 μg/ml, added 1 h before Etoposide) and Etoposide (1 μM, 16 h). After the cells were treated as described above, the total RNA of the cells was extracted, and the relative changes in mRNA levels of BAX, NOXA, PUMA, and P21 in the cells were detected by real-time PCR. The primers used are shown in the following table.

| | Forward | Reverse |
|---|---|---|
| Gene | primer (5′-3′) | primer (5′-3′) |
| BAX | atggacgggtccggggagca | ggtgagcactcccgccacaa |
| | gccc | agat |
| | (SEQ ID NO: 7) | (SEQ ID NO: 8) |
| NOXA | actgttcgtgttcagctc | gtagcacactcgacttcc |
| | (SEQ ID NO: 9) | (SEQ ID NO: 10) |
| PUMA | gacctcaacgcacagta | ctaattgggctccatct |
| | (SEQ ID NO: 11) | (SEQ ID NO: 12) |
| P21 | caccgagacaccactggagg | gagaagatcagccggcgttt |
| | (SEQ ID NO: 13) | (SEQ ID NO: 14) |
| GAPDH | caaggtcatccatgacaact | gtccaccaccctgttgctgt |
| | ttg | ag |
| | (SEQ ID NO: 15) | (SEQ ID NO: 16) |

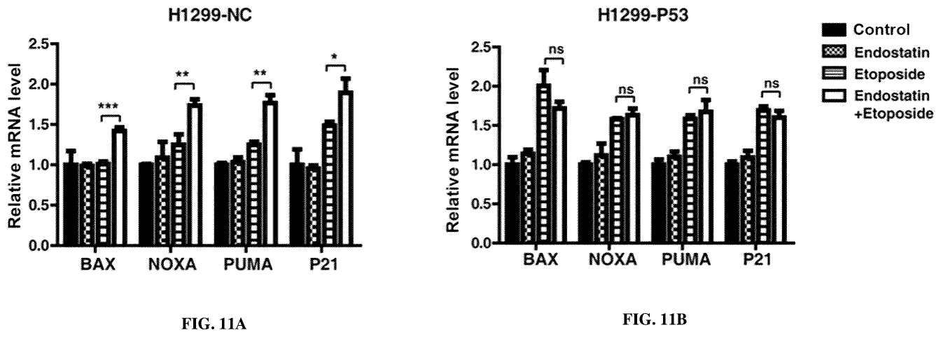

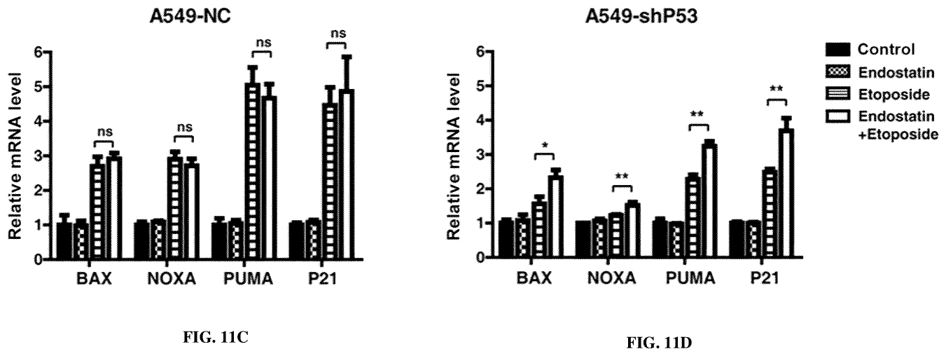

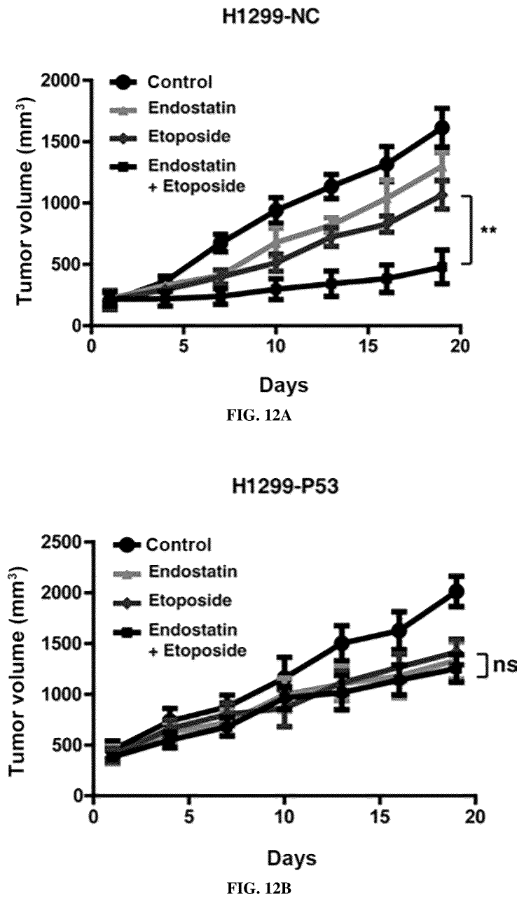

The results showed that for P53-deficient tumor cells (FIGS. 11A and 11D), Endostatin could enhance Etoposide to up-regulate pro-apoptotic molecules. For tumor cells with normal P53 function (FIGS. 11B and 11C), Etoposide could significantly up-regulate the above pro-apoptotic molecules, and Endostatin did not significantly enhance the pro-apoptotic effect of Etoposide.

### Example 12

### Endostatin Enhanced the Inhibitory Effect of Etoposide on the P53-Deficient Tumor Growth

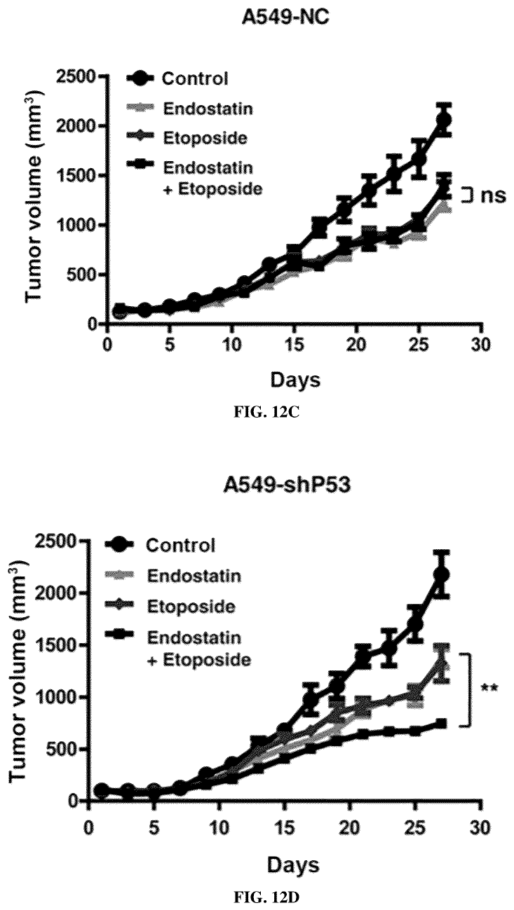

Tumor cells (H1299-NC, H1299-P53, A549-NC, A549-shP53) were inoculated subcutaneously into 6 to 8 week-old nude mice (Beijing Weitong Lihua Experimental Animal Technology Co., Ltd.). Tumor-bearing nude mice were randomly divided into four groups with 6 to 8 animals for each group, and different drug treatments were performed. The first group: negative control; the second group: Endostatin (20 mg/kg, intraperitoneal injection, once a day) treatment; the third group: Etoposide (2 mg/kg, intraperitoneal injection, once every two days) treatment; the fourth group: combined treatment with Endostatin (20 mg/kg, intraperitoneal injection, once a day) and Etoposide (2 mg/kg, intraperitoneal injection, once every two days). Tumor volume was measured every other day. 2 to 4 weeks after administration, the mice were sacrificed and the tumor tissue was removed for further use. The results showed that in the P53-deficient mouse tumor model (FIGS. 12A and 12D), as compared with the control group, the tumor suppression rates of Endostatin were 19.6% (H1299-NC, Day 19) and 37.1% (A549-shP53, Day 27); the tumor suppression rates of Etoposide were 28.0% (H1299-NC, Day 19) and 39.3% (A549-shP53, Day 27); the combination of Endostatin and Etoposide had a synergistic effect on inhibiting tumor growth, with tumor suppression rates of 66.9% (H1299-NC, Day 19) and 65.8% (A549-shP53, Day 27). In a mouse tumor model with normal P53 function (FIGS. 12B and 12C), as compared with the control group, the tumor suppression rates of Endostatin were 33.8% (H1299-P53, Day 19) and 40.9% (A549-NC, Day 27); the tumor suppression rates of Etoposide were 30.0% (H1299-P53, Day 19) and 34.0% (A549-NC, Day 27); Endostatin could not enhance the inhibitory effect of Etoposide on tumor growth, and the tumor suppression rates of the combination were 37.7% (H1299-P53, Day 19) and 32.2% (A549-NC, Day 27).

### Example 13

### The Combined Use of Endostatin and Etoposide had a Synergistic Effect on Prolonging the Survival of P53-Deficient Tumor-Bearing Mice

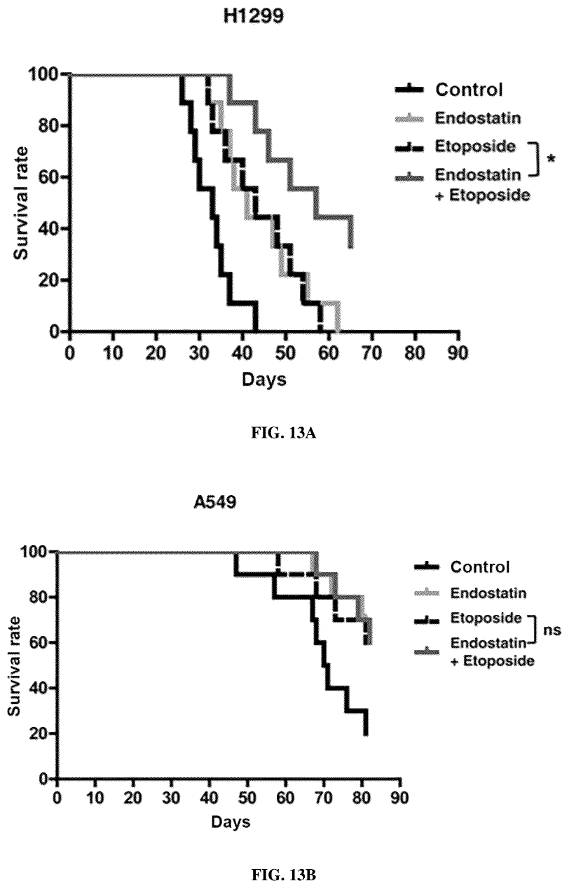

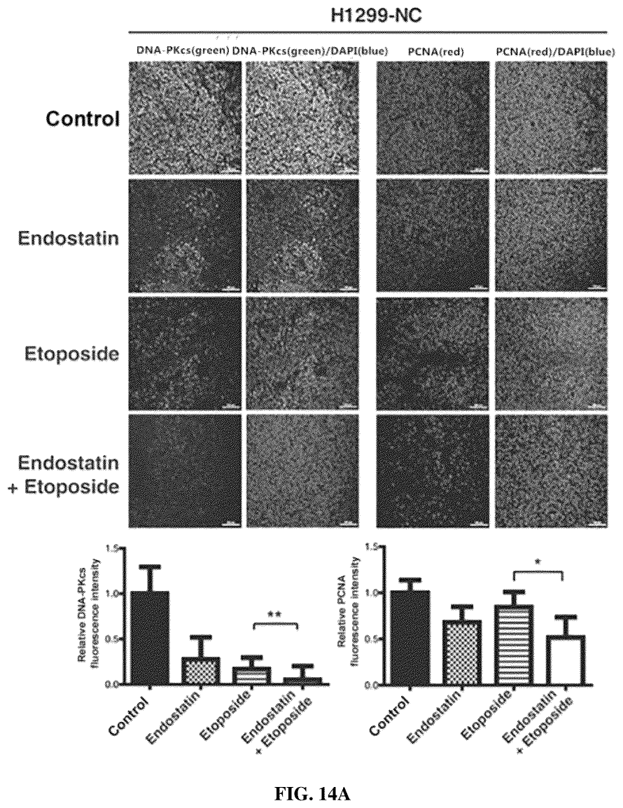

H1299 and A549 cells were cultured in a vigorous and proliferative state, and tumor collection, grouping, and administration methods were the same as in Example 12. The administration was stopped after 25 days, and the survival of the mice was recorded daily, and the survival curve of the mice was plotted. The results showed that in the H1299 mouse tumor model (FIG. 13A), as compared with the control group, the median survival time of the Endostatin-treated group was 24.2% longer than that of the control group; the median survival time of the Etoposide treatment group was 30.3% longer than that of the control group; the combination of Endostatin and Etoposide had a synergistic effect on prolonging the survival time of mice, and the median survival time was 72.7% longer than that of the control group. In a mouse tumor model with normal P53 function (FIG. 13B), as compared with the control group, the Endostatin treatment group and the Etoposide treatment group could prolong the survival time of the mice to some extent, and the combination of Endostatin and Etoposide had no synergistic effect on prolonging the survival time of mice.

### Example 14

### Endostatin Synergized with Etoposide Inhibited the Proliferation of P53-Deficient Tumors and the Expression Level of DNA-PKcs

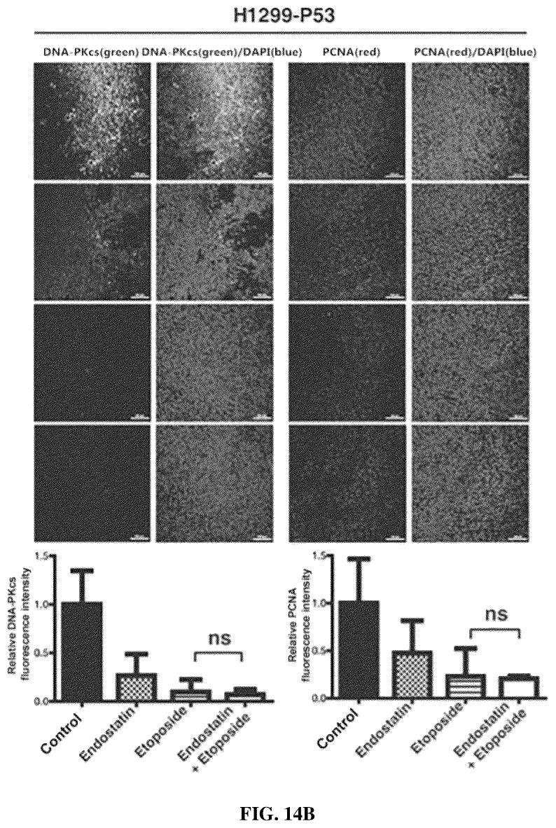

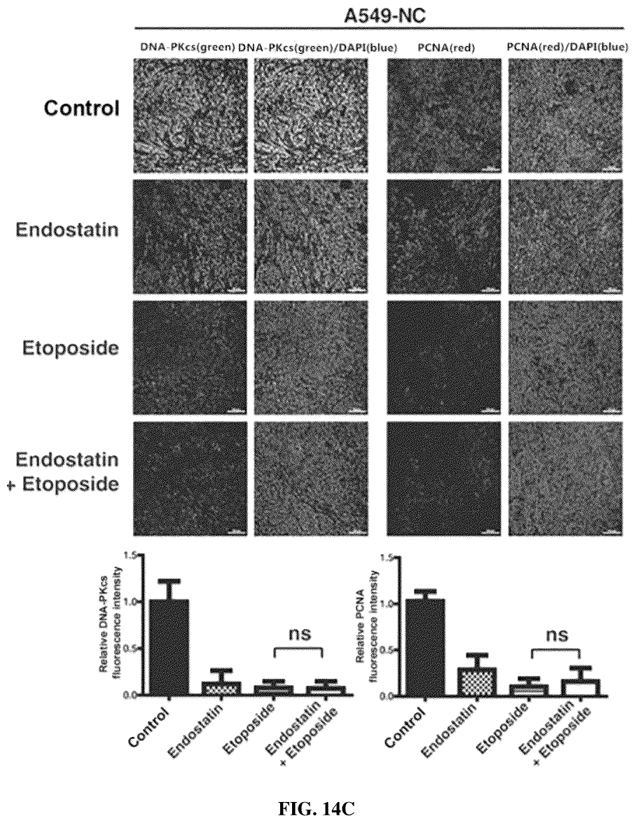

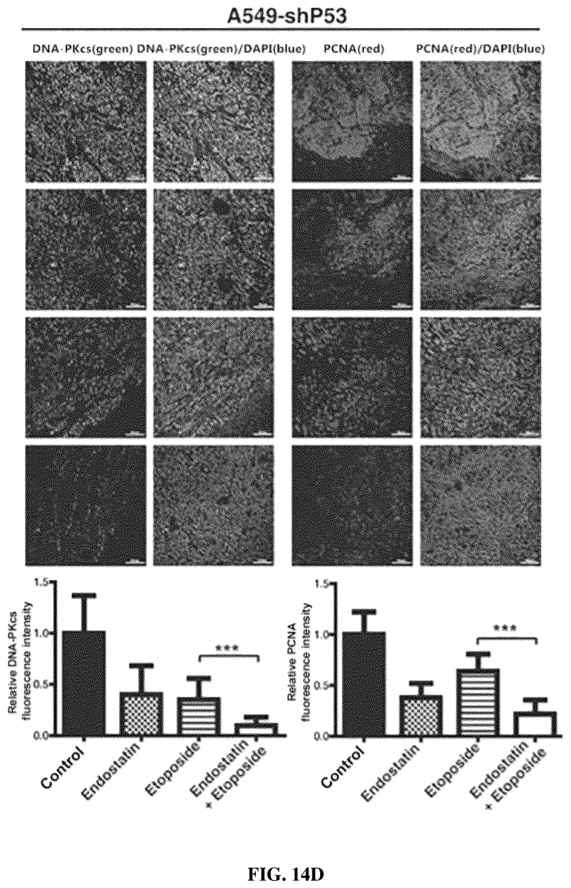

The tumor tissue removed in Example 12 was fixed and embedded, and then sectioned. PCNA (a marker molecule indicating the vigorous proliferation status of tumor cells) and DNA-PKcs protein in tumor tissues were stained by immunofluorescence technique, and were observed and imaged with a laser confocal microscope (Nikon A1). The experimental results are shown in FIG. 14A-14D, in which blue represents the nucleus of DAPI-stained cells, green represents the DNA-PKcs protein, and red represents the PCNA protein. Taking the H1299-NC tumor tissue shown in FIG. 14A as an example, the first column on the left represents DNA-PKcs single staining, and the green area represents the DNA-PKcs protein expression level. The second column on the left represents the results of DNA-PKcs (green) merged with nucleus (blue), and the overlapping part is shown in cyan. The third column on the left represents PCNA single staining, and the red area represents the PCNA protein expression level, indicating the strength of tumor cell proliferation. The fourth column on the left represents the result of the PCNA (red) merged with nucleus (blue), and the overlapping part is shown in purple. Similarly, FIGS. 14B, 14C, and 14D show the expression of PCNA and DNA-PKcs in H1299-P53, A549-NC, and A549-shP53 tumor tissues, respectively. The order of protein staining and image arrangement in the figure is consistent with FIG. 12A. Quantitative results showed that in the P53-deficient mouse tumor model (FIGS. 14A and D), as compared with the control group, both Endostatin and Etoposide can attenuate tumor cell proliferation to a certain extent and reduce the protein level of DNA-PKcs; the combination of Endostatin and Etoposide can function synergistically, and can more significantly inhibit tumor proliferation and DNA-PKcs protein levels as compared with the Etoposide treatment group. In a mouse tumor model with normal P53 function (FIGS. 14B and 14C), as compared with the control group, Endostatin, Etoposide, and the combination of Endostatin and Etoposide could reduce the proliferation of tumor cells and reduce the protein level of DNA-PKcs, and Endostatin did not enhance the inhibitory effect of Etoposide on tumor proliferation and the effect of Etoposide on the protein levels of DNA-PKcs.

### Example 15

### ZBP-Endostatin and PEG-Endostatin Inhibited the Activation of DNA-PKcs Signaling Pathway in Tumor Cells Induced by a Chemotherapeutical Agent

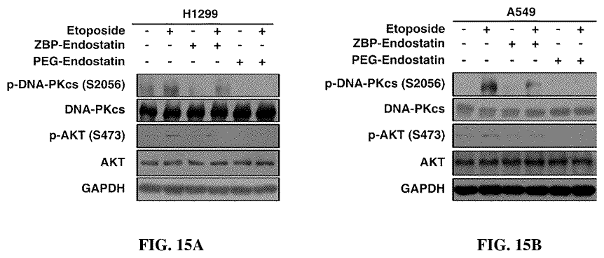

In this example, the inhibition of the DNA-PKcs pathway by ZBP-Endostatin and PEG-Endostatin was verified by detecting the activation levels of DNA-PKcs and AKT. Tumor cells (including H1299 and A549 cells) were divided into six treatment groups. The first group: negative control; the second group: Etoposide (10 μM, 1 h) treatment; the third group: ZBP-Endostatin (10 μg/ml, 1 h) treatment; the fourth group: combined treatment with ZBP-Endostatin (10 μg/ml, added 1 h before Etoposide) and Etoposide (10 μM, 1 h); the fifth group: PEG-Endostatin (10 μg/ml, 1 h) treatment; the sixth group: combined treatment with PEG-Endostatin (10 μg/ml, added 1 h before Etoposide) and Etoposide (10 μM, 1 h). Immunoblotting assay results showed (FIG. 15A-15B) that as compared with the control group, Etoposide treatment activated the level of the DNA-PKcs signaling pathway in H1299 and A549 cells (as shown in the immunoblotting assay results of p-DNA-PKcs (S2056) and p-AKT (S473), wherein p-DNA-PKcs (S2056) represents the phosphorylation level of DNA-PKcs, and S2056 represents its phosphorylation site, wherein p-AKT represents the phosphorylation level of AKT, and S473 represents its phosphorylation site); the combination of ZBP-Endostatin or PEG-Endostatin and Etoposide inhibited the activation of the DNA-PKcs signaling pathway by Etoposide.

1. A method of increasing sensitivity of a cell to a chemotherapeutic agent that induces DNA double-strand break, comprising a step of contacting the cell with an endostatin, wherein the cell is deficient in P53 function.

2. The method according to claim 1, wherein the cell is a tumor cell or cancer cell.

3. The method according to claim 2, wherein the tumor cell or cancer cell is a non-small cell lung cancer cell or melanoma cell.

4. The method according to claim 1, wherein the method is performed in vivo or in vitro.

5. The method according to claim 1, wherein the step of contacting the cell with an endostatin is performed simultaneously or sequentially with the administration of the chemotherapeutic agent.

6. The method according to claim 5, wherein the cell is contacted with an endostatin prior to or after the chemotherapeutic agent.

7. The method according to claim 1, wherein the chemotherapeutic agent is Etoposide or Doxorubicin.

8. The method according to 1, wherein the endostatin is:
a natural human endostatin;
an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or
a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

9. A method of treating tumor or cancer in a subject, comprising:
a) administering a chemotherapeutic agent that induces DNA double-strand break in the subject; and
b) administering an endostatin to the subject,
wherein the tumor or cancer is deficient in P53 function.

10. The method according to claim 9, wherein a therapeutic dose of the treatment regimen for inducing DNA double-strand break is less than a therapeutic dose of the treatment regimen when used alone.

11. The method according to claim 9, wherein the tumor or cancer is non-small cell lung cancer or melanoma.

12. The method according to claim 9, wherein the step of contacting the cell with an endostatin is performed simultaneously or sequentially with the administration of the chemotherapeutic agent.

13. The method according to claim 12, wherein an endostatin is administered to the subject prior to or after the chemotherapeutic agent.

14. The method according to claim 9, wherein the chemotherapeutic agent is Etoposide or Doxorubicin.

15. The method according to claim 9, wherein the subject is a human.

16. The method according to claim 9, wherein the endostatin is:
a natural human endostatin;
an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or
a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.

17. A method for inducing apoptosis, including:
a) inducing DNA double-strand break in a cell that is deficient in P53 function; and
b) contacting the cell with an endostatin;
wherein the DNA double-strand break is induced by contacting the cell with a chemotherapeutic agent.

18. The method according to claim 17, wherein a dosage of the chemotherapeutic agent applied in the step of inducing DNA double-strand break in a cell is less than a dosage applied when the chemotherapeutic agent is used alone.

19. The method according to claim 17, wherein the chemotherapeutic agent is Etoposide or Doxorubicin.

20. The method according to claim 17, wherein the endostatin is:
a natural human endostatin;
an endostatin variant obtained by adding 9 additional amino acids MGGSHHHHH (SEQ ID NO: 1) to the N-terminus of the natural human endostatin, wherein the Met at the N-terminus of the endothelin variant is sometimes partially deleted when expressed by E. coli; or
a product obtained by modifying a natural human endostatin with a monomethoxy polyethylene glycol propionaldehyde (mPEG-ALD) with a molecular weight of 20 kDa, wherein their coupling site is the activated mPEG-ALD aldehyde group and the N-terminal α-amino group of the natural human endostatin.


SEQUENCE LISTING (16 sequences)

SEQ ID NO: 1 - protein, 9 aa, synthetic construct
  REGION 1..9: note = Amino acid fragment
MGGSHHHHH

SEQ ID NO: 2 - protein, 184 aa, synthetic construct
  REGION 1..184: note = The amino acid sequence of natural human endostatin
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 3 - protein, 192 aa, synthetic construct
  REGION 1..192: note = The amino acid sequence of ZBP-Endostatin
MGGSHHHHHHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 4 - DNA, 19 bp, synthetic construct
  misc_feature 1..19: note = P53 sequence
  source 1..19: mol_type = other DNA
gactccagtggtaatctac

SEQ ID NO: 5 - DNA, 21 bp, synthetic construct
  misc_feature 1..21: note = The forward siRNA sequence of DNA-PKcs
  source 1..21: mol_type = other DNA
cagggtttaatcagaatattt

SEQ ID NO: 6 - DNA, 21 bp, synthetic construct
  misc_feature 1..21: note = The reverse siRNA sequence of DNA-PKcs
  source 1..21: mol_type = other DNA
atattctgattaaaccctgtt

SEQ ID NO: 7 - DNA, 24 bp, synthetic construct
  misc_feature 1..24: note = Forward primer of BAX
  source 1..24: mol_type = other DNA
atggacgggtccggggagcagccc

SEQ ID NO: 8 - DNA, 24 bp, synthetic construct
  misc_feature 1..24: note = Reverse primer of BAX
  source 1..24: mol_type = other DNA
ggtgagcactcccgccacaaagat

SEQ ID NO: 9 - DNA, 18 bp, synthetic construct
  misc_feature 1..18: note = Forward primer of NOXA
  source 1..18: mol_type = other DNA
actgttcgtgttcagctc

SEQ ID NO: 10 - DNA, 18 bp, synthetic construct
  misc_feature 1..18: note = Reverse primer of NOXA
  source 1..18: mol_type = other DNA
gtagcacactcgacttcc

SEQ ID NO: 11 - DNA, 17 bp, synthetic construct
  misc_feature 1..17: note = Forward primer of PUMA
  source 1..17: mol_type = other DNA
gacctcaacgcacagta

SEQ ID NO: 12 - DNA, 17 bp, synthetic construct
  misc_feature 1..17: note = Reverse primer of PUMA
  source 1..17: mol_type = other DNA
ctaattgggctccatct

SEQ ID NO: 13 - DNA, 20 bp, synthetic construct
  misc_feature 1..20: note = Forward primer of P21
  source 1..20: mol_type = other DNA
caccgagacaccactggagg

SEQ ID NO: 14 - DNA, 20 bp, synthetic construct
  misc_feature 1..20: note = Reverse primer of P21
  source 1..20: mol_type = other DNA
gagaagatcagccggcgttt

SEQ ID NO: 15 - DNA, 23 bp, synthetic construct
  misc_feature 1..23: note = Forward primer of GAPDH
  source 1..23: mol_type = other DNA
caaggtcatccatgacaactttg

SEQ ID NO: 16 - DNA, 22 bp, synthetic construct
  misc_feature 1..22: note = Reverse primer of GAPDH
  source 1..22: mol_type = other DNA
gtccaccaccctgttgctgtag